(19) United States
(12) Patent Application Publication
(10) Pub. No.: US 2021/0299192 A1
(43) Pub. Date: Sep. 30, 2021

(54) COMPOSITION FOR ANTI-OXIDATION, ORAL HEALTH, IMMUNITY REGULATION AND EXERCISE PROMOTION, AND USES THEREOF

(71) Applicant: GLAC BIOTECH CO., LTD., Tainan City (TW)

(72) Inventors: Hsieh-Hsun HO, Tainan City (TW); Yi Wei KUO, Tainan City (TW); Jia-Hung LIN, Tainan City (TW); Cheng-Ruei LIU, Tainan City (TW)

(21) Appl. No.: 17/065,040

(22) Filed: Oct. 7, 2020

(30) Foreign Application Priority Data
Mar. 31, 2020 (TW) 109111071

(51) Int. Cl. A61K 35/747; A61K 9/68; A61K 47/26; A61K 9/00; A61P 21/00; A61C 15/04; A61C 19/06; A01K 13/00; A01K 15/02

(52) U.S. Cl. CPC A61K 35/747; A61K 9/0058; A61K 47/26; A61K 9/0095; A01K 15/026; A61C 15/041; A61C 19/063; A01K 13/00; A61P 21/00

(57) ABSTRACT

A composition including a strain of Lactobacillus salivarius SA-03 has at least one of the physiologically active effects of anti-oxidation, oral health, immunity regulation, and promotion of exercise. Therefore, the above-mentioned composition can be used for the foregoing physiological activity and is used in a form of food, medicine, and oral cleaning composition.

### BACKGROUND OF THE INVENTION

### 1. Field of the Invention

The present invention relates to a composition and uses thereof, particularly to a composition for at least one of anti-oxidation, oral health, immunity regulation and exercise promotion, and uses thereof.

### 2. Description of the Prior Art

Probiotics are important symbiotic bacteria in human bodies, performing various physiological functions according to different strains and species, including regulation of digestive tract bacteria flora, regulation of digestive tract functions, blood glucose regulation, immunity enhancement, and anti-fatigue. For an example, Lactobacillus rhamnosus GG (LGG) is the probiotic strain studied most frequently so far, having many functions, including balancing and improving gastrointestinal functions, enhancing immunity of human bodies, preventing and healing diarrhea, reducing the risk of respiratory tract infections, discharging toxins, and preventing dental caries. For another example, Bifidobacterium lactis BB-12 has functions of regulating immunity systems, modifying intestinal functions, and inhibiting pathogens. Therefore, the discovery of these multifunctional probiotics is much helpful to human health.

Many recent researches show that variation of the intestinal tract bacteria flora is highly correlated with metabolic diseases and metabolic syndromes. For example, the numbers of the colonies of Bacteroides, Clostridium and E. coli significantly increase in the intestinal microbiota of diabetic patients; the numbers of the colonies of Lactobacillus and Bifidobacterium significantly decrease in the intestinal microbiota of patients of chronic kidney diseases, and probiotic supplement can effectively delay the progression of diseases. Therefore, taking multifunctional probiotics can delay disease progression as well as decrease complication risk.

In general, lactic acid bacteria are safe to human bodies and favorable to health. It has been a target the manufacturers are eager to achieve: developing nutritional supplements containing multifunctional probiotics, which are safe to human bodies and suitable to use persistently.

### SUMMARY OF THE INVENTION

The present invention provides a composition with a strain of a lactic acid bacterium, which has at least one of the physiologically active effects: anti-oxidation, oral health, immunity regulation and exercise promotion. Therefore, the composition with the strain of the lactic acid bacterium of the present invention has the abovementioned physiologically active effects and may be in form of foods, medicine, or oral cleaning agents.

In one embodiment, the present invention provides a composition having multiple physiologically active effects comprises an isolated lactic acid bacteria strain and an excipient, diluent or carrier. The lactic acid bacteria strain includes an SA-03 strain of Lactobacillus salivarius, and the SA-03 strain of Lactobacillus salivarius is deposited in a Deposition No. of CGMCC No. 19519 in China General Microbiological Culture Collection Center (CGMCC).

In one embodiment, the present invention provides a use of a composition with a strain of a lactic acid bacterium for at least one of anti-oxidation, oral health, immunity regulation and exercise promotion comprising administering to a subject the composition, wherein the composition includes an isolated lactic acid bacteria strain and an excipient, diluent or carrier. The lactic acid bacteria strain includes an SA-03 strain of Lactobacillus salivarius, and the SA-03 strain of Lactobacillus salivarius is deposited in a Deposition No. of CGMCC No. 19519 in China General Microbiological Culture Collection Center (CGMCC).

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 shows the results of the experiments for determining the free radical eliminating ability of the lactic acid bacteria strain of the present invention;

FIG. 2 shows the results of the experiments for determining the activity of the superoxide dismutase generated by intestinal epithelial cells (Caco-2 cells) after the co-culture of the Caco-2 cells and the lactic acid bacteria strain of the present invention;

FIG. 3 shows the results of the experiments for determining the activity of the catalase generated by the intestinal epithelial cells (Caco-2 cells) after the co-culture of Caco-2 cells and the lactic acid bacteria strain of the present invention;

FIG. 4 shows the results of the experiments of the ability of the lactic acid bacteria strain of the present invention to inhibit oral pathogens;

FIG. 5 shows the results of the experiments of airway hyperresponsiveness for verifying the ability of the lactic acid bacteria strain of the present invention to inhibit the asthma induced by plasticizer and OVA;

FIG. 6 shows the results of the experiments of the compositions of immune cells for verifying the ability of the lactic acid bacteria strain of the present invention to inhibit the asthma induced by plasticizer and OVA;

FIG. 7 shows the results of the experiments for verifying the effect of the lactic acid bacteria strain of the present invention on generation of antibodies;

FIG. 8 shows the results of the experiments for verifying the effect of the lactic acid bacteria strain of the present invention on regulating the immune-response cytokines of the Treg cells;

FIG. 9 shows the pathological sections of lungs of the mice fed with the lactic acid bacteria strain of the present invention and induced to suffer asthma;

FIG. 10 shows the results of the experiments for verifying the effect of the lactic acid bacteria strain of the present invention on the forelimb grip strength of the mice;

FIG. 11 shows the results of the experiments for verifying the effect of the lactic acid bacteria strain of the present invention on endurance of the mice in the exhaustive swimming tests;

FIG. 12 shows the results of the experiments for verifying the effect of the lactic acid bacteria strain of the present invention on the variation of BUN (blood urea nitrogen) concentrations and CK (creatine kinase) activities after a single cycle of 90-minute swimming and a 60-minute break; and

FIG. 13 shows the results of the experiments for verifying the effect of the lactic acid bacteria strain of the present invention on the glycogen contents in livers and muscles.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The freeze-dried culture of the lactic acid bacterium strain mentioned in the specification is deposited in China General Microbiological Culture Collection Center (CGMCC) of Chinese Academy of Sciences (NO. 1 West Beichen Road, Chaoyang District, Beijing 100101, China). The details thereof are listed in Table. 1.

TABLE 1
Data of Deposited Lactic Acid Bacterium Strains

| Strain | Specie | Deposition No. | Deposition Date |
|---|---|---|---|
| SA-03 | Lactobacillus | CGMCC | Mar. 30, 2020 |
| | salivarius | No. 19519 | |

It is found: the deposited SA-03 strain of Lactobacillus salivarius listed in Table. 1 has at least one of physiologically active effects: anti-oxidation, oral health (such as inhibiting oral pathogens), immunity regulation, anti-allergy/asthma, exercise promotion, and anti-fatigue. Therefore, the deposited Lactobacillus salivarius listed in Table. 1 may be applied to at least one use of anti-oxidation, oral health, immunity regulation, anti-allergy/asthma, exercise promotion, and anti-fatigue.

In one embodiment, the composition having multiple physiologically active effects of the present invention comprises an isolated SA-03 strain of Lactobacillus salivarius and an excipient, diluent or carrier. The SA-03 strain of Lactobacillus salivarius is deposited in a Deposition No. of CGMCC No. 19519 in China General Microbiological Culture Collection Center (CGMCC) of Chinese Academy of Sciences.

In one embodiment, the excipient, diluent or carrier is a physiologically-acceptable excipient, diluent or carrier; thus, the composition of the present invention may be used as a food composition or an oral-cleaning composition. In one embodiment, the excipient, diluent or carrier is a pharmaceutically-acceptable excipient, diluent or carrier; thus, the composition of the present invention may be used as a pharmaceutical composition.

In the embodiment of a food composition, the physiologically-acceptable excipient, diluent or carrier may be a food. The food may be but is not limited to be dairy food, tea, coffee, a tooth-cleaning candy (such as an oral strip, a chewable tablet, or jelly sweets), a functional drink, or a combination thereof. The dairy food may be fermented milk, yoghurt, cheese, milk drink, or powdered milk.

In the embodiment of a pharmaceutical composition, the pharmaceutical composition may be in form of an oral dosage. For example, the oral dosage may be in form of a tablet, a capsule, a solution, or a powder.

In the embodiment of an oral dosage, the excipient or diluent may be tooth paste, mouthwash, tooth powder, mouthwash, a breath freshening spray, a fluoridizing agent (such as a fluoridizing agent smeared on the teeth of children), a false-tooth cleaning agent, a pet tooth cleaning gum, or a hairball remedy gel. The carrier may be a tooth brush, an interdental brush, dental floss, an oral swabstick, or a pet dental chew bone.

In one embodiment, the lactic acid bacteria strain of the present invention is an active strain. In the embodiments of a food composition, an oral cleaning composition or a pharmaceutical composition, the number of the lactic acid bacteria strains may be over 10⁶ CFU (Colony-Forming Unit), preferably over 10¹⁰ CFU.

### Embodiment I: Morphology and General Properties of the Strain of the Present Invention

The taxonomic characteristics of the strain are identified with the 16S rDNA sequencing analysis and the API bacterial identification system. The morphology and general properties of the strains are listed in Table. 2.

TABLE 2
Morphology and General Properties of Lactic Acid Bacteria
Strain of the Present Invention

| Strain | Morphology and characteristics |
|---|---|
| SA-03 strain of | 1. They are gram-positive bacilli, unlikely to |
| Lactobacillus | generate spores, free of catalase, oxidase and |
| salivarius | motility, able to grow in aerobic and anaerobic |
| | environments, most suitable to grow at a |
| | temperature of 37 ± 1° C. They belong to |
| | facultative heterofermentative strains and do |
| | not generate gas in glucose metabolism. |
| | 2. The colonies grown in MRS agar are in form |
| | of solid circles in white color. The bacterium |
| | body has a short rod-like shape, and two ends |
| | thereof are round. The bacterium bodies |
| | normally appear in single bodies. |

### Embodiment II: Collection of the Lactic Acid Bacteria Strain of the Present Invention

The lactic acid bacteria strain of the present invention is preserved in 20% glycerol at a temperature of −80° C. Before use, the strain is activated twice with MRS broth (DIFCO) containing 0.05% cysteine at a temperature of 37° (I for 24 hours. In the present invention, the SA-03 strain of Lactobacillus salivarius is separated from human intestines. In one embodiment, the carbon source in the liquid medium suitable for the SA-03 strain of Lactobacillus salivarius of the present invention may be glucose, fructose, lactose, sucrose, maltose, galactose, mannose, trehalose, starch, molasses, potato starch, corn starch, malt extract, maltodextrin or a combination thereof. For example, the liquid medium used in the preset invention includes 2-5% of a mixture of glucose and maltodextrin, preferably 3% of a mixture of glucose and maltodextrin. In one embodiment, the nitrogen source in the liquid medium suitable for the SA-03 strain of Lactobacillus salivarius of the present invention may be (NH₄)₂SO₄, (NH₄)₃PO₄, NH₄NO₃, NH₄Cl, casamino acid, urea, peptone, polypeptone, tryptone, meat extract, yeast extract, yeast powder, milk, soybean flour, whey, or a combination thereof. For example, the liquid medium used in the preset invention includes at least one of 5-30% milk and 1-10% soybean flour.

### Embodiment III: Analysis of the Free Radical Eliminating Ability of the Lactic Acid Bacteria Strain

DPPH (di(phenyl)-(2,4,6-trinitrophenyl) iminoazanium) is a stable free-radical molecule. DPPH free radicals in methanol have a maximum absorbance at a wavelength of 517 nm. While DPPH free radicals react with anti-oxidation materials, the anti-oxidation materials provide hydrogen ions (protons) to eliminate the free radicals. Thus, the ianthinus color of the DPPH free radicals decays, and the absorbance at 517 nm decreases. The value of OD₅₁₇ is used to determine the free radical eliminating ability of the tested lactic acid bacteria strain.

The method to determine the free radical eliminating ability of the lactic acid bacteria strain is introduced as follows. By a ratio of 1:1, respectively mix 0.2 mM DPPH methanol solutions homogeneously with the following liquids: a suspension of the SA-03 strain of Lactobacillus salivarius of the present invention at a concentration of about 2×10⁹ CFU (about OD=2); a vitamin C solution at a concentration of 4 μg/ml (used as a positive control group); a suspension of the SY-66 strain (free of anti-oxidant activity) of Streptococcus thermophiles at a concentration of about 2×10⁹ CFU (about OD=2)(used as a negative control group); and double-distilled water (used as a blank group). Next, let the mixture liquids react in the dark at an ambient temperature for 30 minutes. Next, the mixture liquids are centrifuged at a speed of 12000 rpm at a temperature of 4° C. for 2 minutes. Next, take 200 μl of liquid from the mixture liquids to a 96-well plate, and measure the values of OD₅₁₇ thereof. The equation for calculating the free radical eliminating ability is expressed as

Free Radical Eliminating Ability=OD_{blank}−ODₛₐₘₚₗₑ/OD_{blank}*100%

wherein ODₛₐₘₚₗₑ is the absorbance of the tested sample and OD_{Blank} is the absorbance of the blank group.

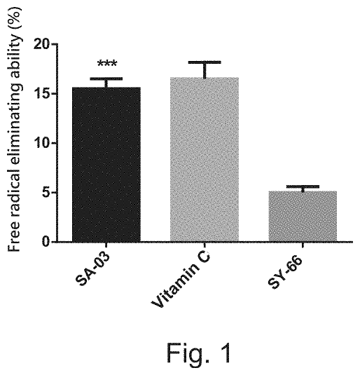

FIG. 1 shows the results of the experiments for determining the free radical eliminating ability of the lactic acid bacteria strain of the present invention (the DPPH assay), wherein *** expresses p<0.005, indicating a high degree of statistical significance. In comparison with the SY-66 strain of Streptococcus thermophiles, the SA-03 strain of Lactobacillus salivarius of the present invention has higher free radical eliminating ability.

### Embodiment IV: Analysis of the Ability of the Lactic Acid Bacteria Strain of the Present Invention to Induce the Intestinal Epithelial Cells to Express Anti-Oxidation Enzymes

The human body has a mechanism to regulate too high oxidative stress. While the oxidative stress is too high, the body will generate antioxidants to deal with such a condition. For example, the body will synthesize glutathione, ubiquinol and uric acid to absorb free electrons. Besides, the antioxidants absorbed from food, such as Vitamin C and Vitamin E, can also inhibit generation of free radicals. Further, the human body has an anti-oxidation system, i.e. the anti-oxidation enzyme system. Superoxide dismutases (SOD) are important anti-oxidation enzymes, able to convert superoxide into oxygen and hydrogen peroxide through disproportionation reactions. The human body has three kinds of superoxide dismutases, which respectively appear in the external of the cells, in the cytoplasm, and in the mitochondria. There is also an enzyme, called the catalase, able to convert the hydrogen peroxide, which is generated by the superoxide dismutase, into oxygen and water. In the saturation state, a catalase molecule can convert forty-million hydrogen peroxide molecules into oxygen and water per second.

The Caco-2 cells are the epithelial cells of human colon gland cancer. The structure and function of the Caco-2 cell is similar to that of the differentiated intestinal epithelial cell. The Caco-2 cells have microvilli and the enzyme system related to the epithelial cells of the intestinal brush border. Therefore, the Caco-2 cells are extensively used to simulate the in-vivo physiological activities of intestinal cells. In a cell culture system, the Caco-2 cells may grow into a single layer of cells, which are arranged closely, and which not only morphologically resemble intestinal epithelial cells but also have the same endocytosis phenomenon and the tight-junction structure.

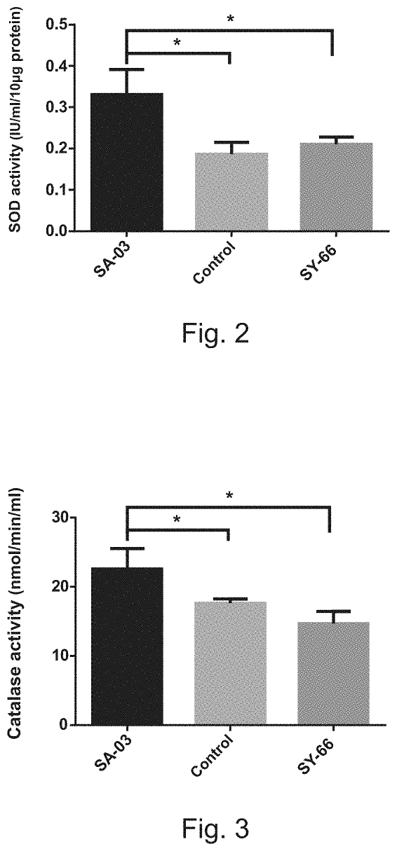

In this experiment, the live SA-03 strain of Lactobacillus salivarius of the present invention (the experiment group), the SY-66 strain of Streptococcus thermophiles (the control group), and a culture solution free of any strain (the control group) are added to the Caco-2 cell culture system by a ratio of cells:probiotics=1:100. The live bacteria strains and the cells are co-cultured for 16 hours. Next, wash out the lactic acid bacteria strains. Next, break the Caco-2 cells and extract the protein to detect the activity of superoxide dismutase (SOD) (the experimental results are shown in FIG. 2) and the activity of catalase (the experimental results are shown in FIG. 3). The experiments respectively adopt the SOD Assay Kit (Cayman Cat. 706002) and the Catalase Assay Kit (Cayman Cat. 707002) in the SOD activity analysis and the catalase activity analysis. The experimental processes are undertaken according to the proposals in the manuals of the kits.

Refer to FIG. 2 and FIG. 3 for the effect of the lactic acid bacteria strain of the present invention to induce the intestinal epithelial cells to express anti-oxidation enzymes, wherein * expresses that p<0.05, indicating statistical significance. In comparison with the SY-66 strain of Streptococcus thermophiles, the SA-03 strain of Lactobacillus salivarius of the present invention can more efficiently induce the Caco-2 cells to express anti-oxidation enzymes (SOD and catalase) to decompose excessive free radicals in the body.

### Embodiment V: Analysis of the Ability of the Lactic Acid Bacteria Strain of the Present Invention to Inhibit Oral Pathogens

Streptococcus mutans, Porphyromonas gingivalis, Fusobacterium nucleatum and Aggregatibacter actinomycetemcomitans are notorious oral pathogens. Streptococcus mutans and Fusobacterium nucleatum grow together with other oral microbes to form biological membrane adhering to the periodontal tissue and cause dental caries and dental plaque. Porphyromonas gingivalis generates protease, which may digest collagen and cause periodontitis. Infection of Aggregatibacter actinomycetemcomitans is usually correlated with invasive periodontitis and mouth odor. Nowadays, various antibiotics, mouthwashes and dental gels are used to treat these oral diseases. However, abuse of these drugs may generate antibiotic-resistant oral pathogens. In order to prevent generation of various antibiotic-resistant oral pathogens, it is a better choice: using probiotics to control the growth of oral pathogens and maintain the stability of oral microflora.

The experiment uses the double-agar overlay method to analyze the abilities of the SA-03 strain of Lactobacillus salivarius of the present invention, the TE-3 strain of Lactobacillus reuteri and the SY-66 strain of Streptococcus thermophiles to inhibit oral pathogens. The SA-03 strain of Lactobacillus salivarius of the present invention, the TE-3 strain of Lactobacillus reuteri and the SY-66 strain of Streptococcus thermophiles (about 10⁹ CFU) are respectively absorbed by swabsticks, and the swab sticks are used to respectively draw lines (2 cm in width) on MRS agar plates. The agar plates are cultured in a semi-anaerobic condition for 48 hours to generate 2 cm wide growth areas of the tested strains. Next, add to the agar plates the Tryptic Soy Broth (TSB) at a temperature of 45° C., which is for culturing Streptococcus mutans, Porphyromonas gingivalis and Fusobacterium nucleatum, and the Brain Heart Infusion (BHI) at a temperature of 45° C., which is for culturing Aggregatibacter actinomycetemcomitans. After the agar cures, the oral pathogens (at a concentration of about 10⁷-10⁹ CFU/ml) are inoculated on the surface of agar with swabsticks, and the agar plates are cultured at a temperature of 37° C. for 48 hours. Next, use a semi-quantitative score system (0-3 points) to measure the widths of the inhibition areas to determine the bacteria-inhibiting activities of the SA-03 strain of Lactobacillus salivarius of the present invention, the TE-3 strain of Lactobacillus reuteri and the SY-66 strain of Streptococcus thermophiles. If the width of the inhibition area=0 cm, it scores 0 points; if the width of the inhibition area=1-2 cm, it scores 1 point; if the width of the inhibition area=2-3 cm, it scores 2 points; if the width of the inhibition area>3 cm, it scores 3 points.

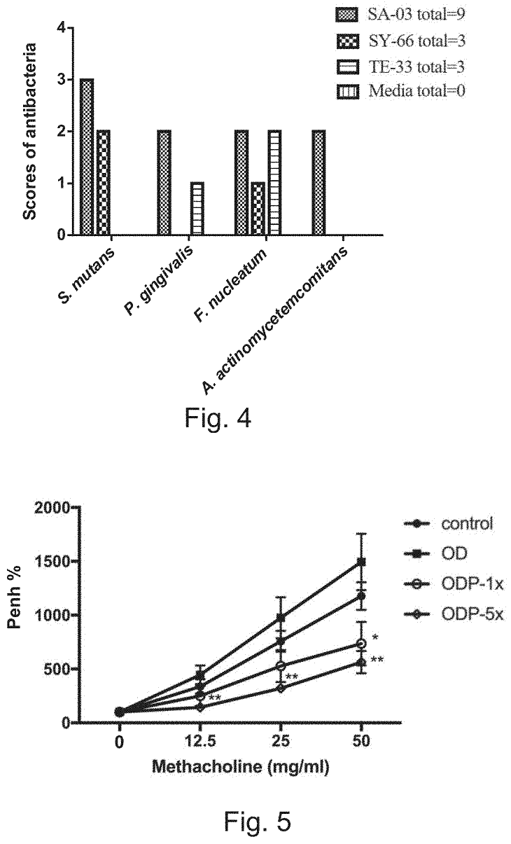

Refer to FIG. 4 for the experimental result of the ability of the SA-03 strain of Lactobacillus salivarius of the present invention to inhibit oral pathogens. The SA-03 strain of Lactobacillus salivarius of the present invention has ability to inhibit all of the oral pathogens: Streptococcus mutans, Porphyromonas gingivalis, Fusobacterium nucleatum and Aggregatibacter actinomycetemcomitans and totally scores 9 points. Each of the SY-66 strain of Streptococcus thermophile and the TE-3 strain of Lactobacillus reuteri only has ability to inhibit a specified oral pathogen and scores only 3 points. In comparison with the SY-66 strain of Streptococcus thermophile and the TE-3 strain of Lactobacillus reuteri the SA-03 strain of Lactobacillus salivarius of the present invention can effectively inhibit the oral pathogens: Streptococcus mutans, Porphyromonas gingivalis, Fusobacterium nucleatum and Aggregatibacter actinomycetemcomitans and maintain oral health.

### Embodiment VI: Analysis of the Ability of the Lactic Acid Bacteria Strain of the Present Invention to Inhibit Allergic Asthma Induced by Plasticizers

In 2011, thousands of children contacted the foods polluted by a plasticizer di(2-ethylhexyl)phthalate (DEHP). In the incident, the cases of childhood allergic asthma obviously increase in clinic. So far, more and more clinic researches have confirmed the relationship between plasticizers and childhood allergic asthma. Plasticizers (phthalate esters) are derivatives generated in esterification of phthalate acid, including DMP, DEP, DPP, DBP, DNPP, DNHP, DCHP, DNOP, DOP, DEHP, DIOP, DNP, DINP, DIDP, and BBP. In general, phthalate esters are aromatic or odorless viscous oily liquid with low volatility and high stability. Phthalate esters are almost insoluble in water but likely to solve in non-polar organic solvents. Phthalate esters have been assumed to be related with allergic diseases, such as asthma and atopic dermatitis.

The experiment uses the plasticizer (DEHP) to induce mice to suffer allergic asthma, whereby to test the ability of the SA-03 strain of Lactobacillus salivarius of the present invention to inhibit allergy and regulate immunity. In the experiment, DEPH is dissolved in olive oil to at a concentration of 400 μg/kg form a DEPH-containing solution. The experiment uses pregnant female BALB/c mice. In the pregnant stage (3 weeks) and the breast-feed stage (3 weeks), tube-feed the mice with the DEPH-containing solution, and tube-feed other mice with pure olive oil to function as a control group. Once the offspring are aged 5-8 weeks, perform an OVA-induced asthma model on the offspring, and feed the offspring with DEPH and the SA-03 strain of Lactobacillus salivarius of the present invention until the animals are sacrificed. In the first day and the fourteenth day, intraperitoneally inject into the offspring 0.1 ml solution containing 20 μg OVA and 2.25 mg Al(OH)₃ together with an adjuvant of phosphate buffered saline (PBS) to function as an allergen and induce whole-body allergy. After two cycles of intraperitoneal injections inducing whole-body allergy, provide the mice with vaporized OVA for three successive days from the twenty-eighth days, wherein the respiratory tracts of the mice are sensitized via letting the mice inhale the vapor containing 1% OVA. The experimental process of the control group is the same as that of the experimental group except OVA is replaced by saline. In the experiment, the animals are divided into four groups each having 6 mice: the control group (C), the OVA/DEHP group (OD), the OVA/DEHP/low-dosage probiotics (the SA-03 strain of Lactobacillus salivarius of the present invention) group (ODP-1×), the OVA/DEHP group (OD), the OVA/DEHP/high-dosage probiotics (the SA-03 strain of Lactobacillus salivarius of the present invention) group (ODP-5×). The amount of bacteria fed to each mouse in the ODP-1× group is 2.05×10⁹ CFU. The amount of bacteria fed to each mouse in the ODP-5× group is 1.03×10¹⁰ CFU. In the thirty-first day, undertake an airway hyperresponsiveness (AHR) test. In the next day, after sampling and sacrifice, undertake the analysis of allergic factors in serum, the analysis of pathological sections of lungs, and the analysis of cells and cytokines of the lung lavage fluid. The details of the abovementioned experiments are described as follows.

### Airway Hyperresponsiveness (AHR) Test

Methacholine is a non-specific bronchoconstrictor, which acts on the muscarinic receptors of the parasympathetic plexus of the bronchial. Once a potential asthma patient inhales the spray of methacholine, the smooth muscle of the bronchial will constrict, and the person will suffer asthma. Therefore, the sensitivity of the lungs of the animals may be evaluated after the tested animals inhale the spray of methacholine. In the thirty-second day, after OVA induction, undertake the airway hyperresponsiveness (AHR) test of the mice to observe the constriction of the respirator tracts of the mice. Firstly, expose the mice to different concentrations of methacholine (Sigma-Aldrich, Inc.), respectively 0, 12.5, 25 and 50 mg/ml in form of spray for 3 minutes. Next, respectively place the mice in a chamber equipped with an AHR detection system (BUXCO Electronics, Inc., Wilmington, N.C., USA) for 3 minutes. The sensors will detect respiratory rate of the mouse and the airflow variation. Then, the software BioSystem XA analyzes the detection values and outputs the Penh value (enhanced pause). The higher the Penh value, the more serious the asthma.

### Analysis of Immune Cells

Firstly, sample the blood of the sacrificed animals. Next, place the sampled blood in collection tubes containing an anticoagulant EDTA, and mix them homogeneously at ambient temperature. Next, use automated hematology analyzer to detect different types of white blood cells (WBC), including neutrophil, lymphocyte, monocyte, eosinophil and basophil. The number of eosinophil is used to evaluate the seriousness of asthma. The smaller the number of eosinophil, the less serious the asthma.

### Analysis of the Concentrations of Specific IgE and IgG1 in Serum

Firstly, apply 10 μg/ml OVA (dissolved in 1×PBS) to a 96 well ELISA plate (TPP, Trasadingen, Switzerland), and place the well plate at a temperature of 4° C. overnight. Next, undertake blocking with 3% bovine serum albumin (BSA) (in PBS) at a temperature of 37° C. One hour later, add the tested material to the well plate, and let it react at a temperature of 37° C. for one hour to form a first semi-product. Next, add biotinylated rat anti-mouse monoclonal IgE or IgG1 (BD Biosciences) to the first semi-product, and let it react at a temperature of 37° C. for one hour to form a second semi-product. Next, add streptavidin conjugated-HRP to the second semi-product, and let it react at ambient temperature for 30 minutes to form a third semi-product. Next, add an acceptor solution, which includes 2 mg o-Phenylenediamine dihydrochloride (OPD, Sigma-Aldrich) and 2 μl 30% H₂O₂ (dissolved in 5 ml PBS), to the third semi-product for coloring, and let it react at ambient temperature for 20 minutes to form a fourth semi-product. Next, use 25 μl 3 M H₂SO₄ to terminate the reaction. Then, use ELISA reader (Sunrise™, TECAN Ltd.) to detect the light absorption at a wavelength of 450 nm. The lower the concentration of IgE, the more unlikely to suffer allergy and asthma.

### Analysis of the Cytokines in Bronchoalveolar Lavage Fluid (BALF)

After the animal experiment ends, use Zoletil 50 (1 mL) to sacrifice the mice, and use a 24G hose needle, a 1 mL syringe and a PE60 hose to inject 1 mL saline into the lungs of the mice. Next, undertake 3 cycles of BALF sucking to recycle 0.5 mL BALF. Next, use the Luminex machine to detect the concentrations of the cytokines IL-4, IL-5, IL-10, IL-13 and IFN-γ in BALF and serum, and use the Milliplex kit to coat the antibody, which is to be analyzed, to microbeads. In principle, the Milliplex kit uses the antibody-antigen immunological bonding to undertake detection. An infrared fluorescent pigment and a far infrared fluorescent pigment are mixed in different ratios to generate 100 color codes. A microbead having a special color code is engaged with an antibody able to specifically identify a given protein. Next, let the microbeads react with the detection antibodies of labelling biotins. Next, add SAPE (Streptavidin Phycoerythrin) to the microbeads to facilitate a fluorescence-antibody reaction. Thus, the concentration of a cytokine can be learned via detecting the intensity of fluorescence. The lower the concentration of a Th2 cytokine (such as IL-4, IL-5, or IL-13), the slighter the allergy. The higher the concentration of IL-10 or IFN-γ, the more unlikely to suffer the allergy and asthma induced by the Th2 immune reaction.

### Analysis of Composition of Immune Cells in BALF

Firstly, acquire the BALF of the sacrificed mice, and use the ACK lysis buffer (Becton Dickinson) to remove red blood cells. Next, suspend the BALF, whose red blood cells have been removed, in the FACS buffer (Becton Dickinson), and undertake coloring with a monoclonal antibody (cd4, cd8, cd3, cd19, or foxp3). Then, use the Accuri C6 flow cytometer (BD Biosciences) to undertake detection, and use the BD Accuri™ C6 software to analyze the composition of the immune cells.

### Analysis of Pathological Sections of Lungs

Firstly, use OVA to induce chronical histopathological changes of lung tissue of the mice. Next, sacrifice the mice, and sample a single left lung lobe from each mouse. Next, transversely take a section from the middle region of each sample, and prepare the sections to be H&E-stained sections and PAS-stained sections for observation. The animal number n=6 is used in each evaluation method. The evaluation method of Wittke et al. (2004) is adopted and moderately modified. In the histopathological evaluation, three different histomorphologic variations are used in quantitative evaluation, including bronchial epithelial cell proliferation, lung inflammation, and bronchial mucus secretion (stained by PAS). The histomorphologic variation may score 0-4 points according to the severity thereof. No response scores 0 points. The most severe condition scores 4 points. Then, the sum of the scores of the three different histomorphologic variations is used as an integral index of asthma.

Refer to FIG. 5 for the results of the experiment of the ability of the SA-03 strain of Lactobacillus salivarius of the present invention to inhibit the asthma induced by plasticizer and OVA. It is learned from FIG. 5: the OD (OVA plus DEHP) group has the highest degree of increase in the Penh value (enhanced pause) in comparison with the group of 0 mg/ml. Under different concentrations of methacholine, the result of the OD group is significantly different from the result of the high dosage group (ODP-5×). The group of 0 mg/ml of methacholine is used as the standard of comparison. While the spray of 12.5 mg/ml methacholine is used, the Penh value of the OD group is 4.46 times the Penh value of the group of 0 mg/ml of methacholine, and the Penh value OF the high dosage group is 1.44 times the Penh value of the group of 0 mg/ml of methacholine. While the spray of 25 mg/ml methacholine is used, the Penh value of the OD group is 9.76 times the Penh value of the group of 0 mg/ml of methacholine, and the Penh value OF the high dosage group is 3.21 times the Penh value of the group of 0 mg/ml of methacholine. While the spray of 50 mg/ml methacholine is used, the Penh value of the OD group is 14.95 times the Penh value of the group of 0 mg/ml of methacholine, and the Penh value of the high dosage group is Penh value times the AHR of the group of 0 mg/ml of methacholine (p<0.0). Therefore, it is learned: in comparison with the Penh value of the OD group, the Penh value of the high dosage (ODP-5×) group and the Penh value of the low dosage (ODP-1×) group are significantly decreased, respectively 0.37 times and 0.5 times the Penh value of the OD group while the spray of the highest dosage of methacholine (50 mg/ml) is used.

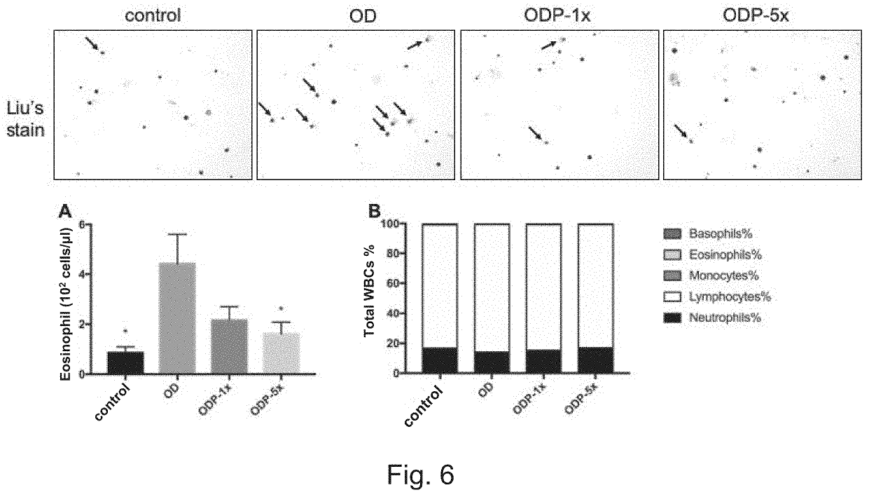

Refer to FIG. 6 for the effect of the SA-03 strain of Lactobacillus salivarius of the present invention on the compositions of immune cells in the experiment of using plasticizer and OVA to induce asthma. 10 μl of the bronchoalveolar lavage fluid (BALF) and 10 μl of the suspension liquid of blood cells are respectively dripped on microslides and processed with Liu staining. Next, count the numbers of eosinophil under a microscope. It is learned from FIG. 6A: the number of eosinophil in the BALF of the OD group is significantly higher than that of other groups. The number of eosinophil in the BALF of the ODP-5× group containing a high dosage of the SA-03 strain of Lactobacillus salivarius is significantly decreased (p<0.05). Collect blood, and use an automated hematology analyzer to detect different types of white blood cells (WBC). It is shown in FIG. 6B: the compositions of the white blood cells, including neutrophil, lymphocyte, monocyte, eosinophil and basophil, of all the groups do not vary in the period of experiments. It is shown by FIG. 6: the SA-03 strain of Lactobacillus salivarius of the present invention doses not regulate the variation of the composition of the systemic immune cells but the mainly regulates the composition of the immune cells in the allergic region. It is easily understood: lowering the number of eosinophil is favorable to alleviate the symptom of asthma.

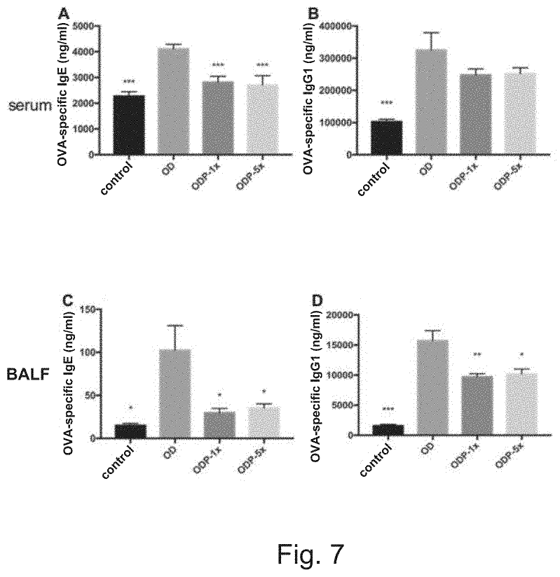

Refer to FIG. 7 for the effect of the SA-03 strain of Lactobacillus salivarius of the present invention on generation of antibodies. After the serums of all the groups are separated, undertake analysis of the immunoglobulins IgE and IgG1, which are related with asthma and specific to OVA. It is shown in FIG. 7A: the concentration of the OVA-specific IgE in the OD group is 4106.5±183.8 ng/ml, which is significantly higher than other groups. In the ODP-1× and ODP-5× groups, which are provided with the SA-03 strain of Lactobacillus salivarius of the present invention, the concentrations of the OVA-specific IgE are significantly lower than the OD group, and the difference between the OD group and the ODP-1×/ODP-5× group is statistically significant (p<0.005). However, no significant difference exists between the low dosage (ODP-1×) group and the high dosage (ODP-5×) group, wherein the concentration of IgE in the ODP-1× group is 2812.7±233.3 ng/ml, and the concentration of IgE in the ODP-5× group is 2700.5±368.2 ng/ml. Refer to FIG. 7C. The concentration of the OVA-specific IgE in BALF is highest in the OD group. The concentrations of the OVA-specific IgE in BALF of the ODP-1× and ODP-5× groups, which are provided with the SA-03 strain of Lactobacillus salivarius of the present invention, are significantly lower than the OD group. The difference of between the OD group and the ODP-1×/ODP-5× group is statistically significant (p<0.05).

Refer to FIG. 7B. The concentration of the OVA-specific IgG1 in the serum of the control group is significantly lower than the OD group, and statistically significant difference exists therebetween (p<0.005), wherein the concentration the OVA-specific IgG1 in the serum of the control group is 102936±7121 ng/ml, and the concentration of the OVA-specific IgG1 of in the serum of the OD group is 325194±53859 ng/ml. Refer to FIG. 7D. In comparison with the control group, the concentration the OVA-specific IgG1 in BALF of the OD group significantly rises, and statistically significant difference exists therebetween (p<0.005), wherein the concentration the OVA-specific IgG1 of in BALF of the control group is 1566.7±238.8, and the concentration the OVA-specific IgG1 in BALF of the OD group is 15709±1668 ng/ml. In comparison with the OD group, the concentrations of the OVA-specific IgG1 in BALF of the ODP-1× and ODP-5× groups, which are provided with the SA-03 strain of Lactobacillus salivarius of the present invention, are significantly lower than the OD group, wherein the concentration the OVA-specific IgG1 in BALF of the ODP-1× group is 9714.5±504 ng/ml (p<0.01), and the concentration the OVA-specific IgG1 in BALF of the ODP-5× group is 10167±840 ng/ml (p<0.05). Therefore, it is learned from FIG. 7: the SA-03 strain of Lactobacillus salivarius of the present invention can effectively decrease the concentrations of the OVA-specific IgE and IgG1 and alleviate the symptom of asthma.

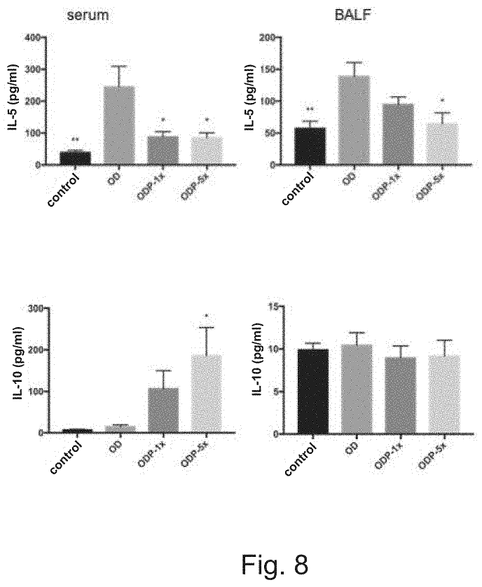

Refer to FIG. 8 for the effect of the SA-03 strain of Lactobacillus salivarius of the present invention on regulating the immune-response cytokines IL-5 and IL-10 of the Treg cells. The SA-03 strain of Lactobacillus salivarius of the present invention may induce the Treg cells to generate the cytokine IL-10. The cytokine IL-10 may inhibit the Th1 and Th2 immune response and thus alleviate allergy and asthma. As shown in FIG. 8, the concentration of the cytokine IL-5 significantly varies in serum and BALF; the concentrations of the cytokine IL-5 in serum and BALF of the OD group rise significantly. The concentration of the cytokine IL-5 in serum of the OD group is significantly higher than the control group (p<0.01). The concentrations of the cytokine IL-5 in serum of the ODP-1× and ODP-5× groups, which are provided with the SA-03 strain of Lactobacillus salivarius of the present invention, are significantly lower than the OD group (p<0.05). On the other side, the concentration of the cytokine IL-10, which can regulate the Th1/Th2 balance, in serum of the ODP-5× group is significantly higher than the OD group (p<0.05). It is learned from FIG. 8: DEHP plus OVA can encourage the activation of the cytokine IL-5 in serum of the lung and the nearby bronchial tissue; supplement of the SA-03 strain of Lactobacillus salivarius of the present invention can inhibit the increase of cytokine IL-5 and alleviate asthma.

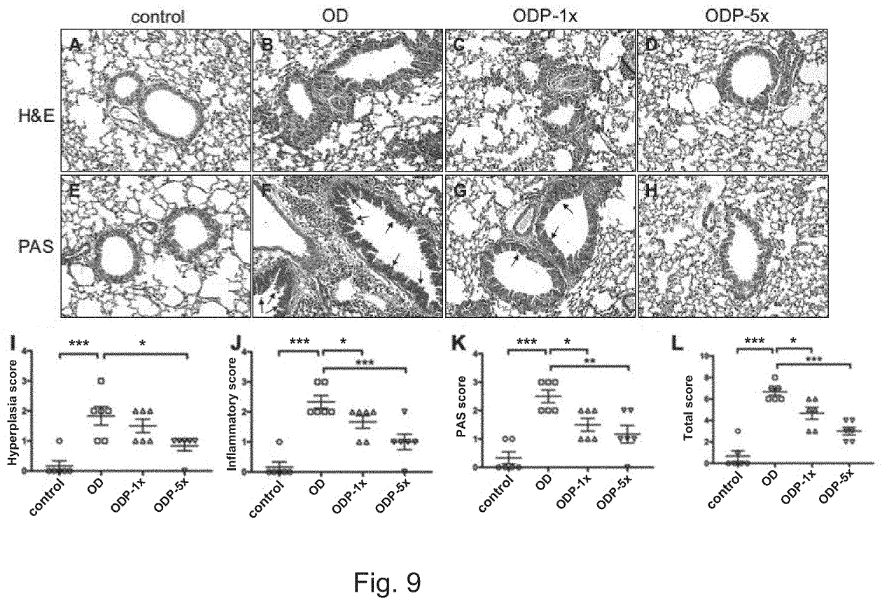

Refer to FIG. 9 for the pathological sections of lungs of the mice fed with the SA-03 strain of Lactobacillus salivarius of the present invention. The pathological sections of lungs of all the groups of mice are respectively shown in FIGS. 9A-9H. In the OD group, proliferation and hypertrophy appear in the epithelial cells of the bronchial tubes; a portion of bronchial tubes and blood vessels are infiltrated by inflammatory cells and thickened, as shown in FIG. 9B. The PAS-staining method is used to evaluate the mucus secretion in goblet epithelial cells of the bronchial tubes. As shown in FIG. 9F, mucus obviously increases in the epithelial tissue of the bronchial tubes of the OD group, wherein the arrows point to the secreted mucus. In contrast to the OD group, proliferation, inflammation, and mucus secretion in the epithelial cells of the bronchial tubes of the lungs is obviously improved in the of the ODP-1× and ODP-5× groups, which are provided with the SA-03 strain of Lactobacillus salivarius of the present invention. Refer to FIGS. 9I-9L. Further, the results of quantitatively analyzing various indexes also show that the abovementioned problems are improved and have statistically significant difference, wherein * indicates p<0.05; ** indicates p<0.01; *** indicates p<0.005.

### Embodiment VII: Analysis of the Effect of the Lactic Acid Bacteria Strain of the Present Invention on Exercise Performance

While an animal is in fatigue, it will fail to achieve the normal performance of action or exercise. Fatigue is a subjective perception, usually related with tissue injury or energy deficiency. Fine intestinal bacteria flora can effectively enhance the exercise performance and anti-fatigue ability of the host.

The experiment uses an exercise mode to analyze the effect of the SA-03 strain of Lactobacillus salivarius of the present invention on enhancing exercise performance and anti-fatigue ability of mice. The design of the experiment refers to the supplement dosage of probiotics recorded in documents and the recommended dietary allowance of human beings. In other words, 1time dosage of probiotic supplement in the experiment is based on that “the recommended daily supplement of probiotics for a person is 1×10¹⁰ CFU”. For an adult weighing 60 kg, the recommended daily intake is 1*10¹⁰ CFU. In order to understand whether a dosage effect occurs in feeding the bacteria strain, 1 time, twice, 5 times the recommended dosage of a person are respectively supplied to different groups. Additionally, a control is used for comparison. The metabolic rate of a human being is different from that of a tested animal. The dosage for a mouse is 12.3 times the dosage for a human being. According to the method of “Estimating the maximum safe starting dose in initial clinical trials for therapeutics in adult healthy volunteers”, 1 time the dosage for a mouse is 2.05×10⁹ CFU/kg/day; twice the dosage for a mouse is 4.10×10⁹ CFU/kg/day; 5 times the dosage for a mouse is 1.03×10¹⁰ CFU/kg/day. The PBS of the same volume is supplied to the blank control group.

Purchase totally 40 six-week-old male ICR mice from BioLASCO Co., Ltd. Provide animal feed and water, and let the mice eat freely. The animal house is controlled to be at a temperature of 24±2° C. and a humidity of 60-70%, illuminated and darkened alternately for 12 hours. Raise the animals for 2 weeks to accommodate themselves to the environment until they become 8-week-old adult mice. Select 40 mice weighing similarly, and randomly divide the mice into 4 groups: (a) the blank control group (Vehicle), (b) the group fed with 1 time the dosage (1×): 2.05×10⁹ CFU/kg mouse/day, (c) the group fed with twice the dosage (2×): 4.10×10⁹ CFU/kg mouse/day, (d) the group fed with 5 times the dosage (5×): 1.03×10¹⁰ CFU/kg mouse/day.

The animal experiment spans 4 weeks. From the first week to the fourth week, daily tube-feed the mice the SA-03 strain of Lactobacillus salivarius of the present invention. After the mice have been continuously supplied with the SA-03 strain of Lactobacillus salivarius of the present invention for 4 successive weeks, analyze the biochemical indexes related with exercise performance and fatigue in sequence. In order to meet the welfare of animals, the intensity of exercise is increased from low to high, and the time of exercise is increased from short to long. Next, draw the blood of the mice for various tests. After different dosages of the SA-03 strain of Lactobacillus salivarius of the present invention have been respectively fed to different groups for 4 weeks, undertake the following exercise experiments and anti-fatigue experiments.

### Experiment of Forelimb Grip Strength

The experiment is to evaluate the effect of different dosage of the SA-03 strain of Lactobacillus salivarius of the present invention on the forelimb grip strength of the mice, whereby to learn the promotion of muscle strength. Thirty minutes later after the mice were fed with the SA-03 strain of Lactobacillus salivarius of the present invention on the twenty-ninth day of the experiment, the experiment of forelimb grip strength starts. The experiment is based on the following papers: (Huang W C, Lin C I, Chiu C C, Lin Y T, Huang W K, Huang H Y, Huang C C. (2014). Chicken essence improves exercise performance and ameliorates physical fatigue. Nutrients. 6(7): 2681-2696); (Wu R E, Huang W C, Liao C C, Chang Y K, Kan N W, Huang C C. (2013). Resveratrol protects against physical fatigue and improves exercise performance in mice. Molecules 18(4):4689-4702); (Yeh T S, Chuang H L, Huang W C, Chen Y M, Huang C C, Hsu M C. (2014). Astragalus membranaceus Improves Exercise Performance and Ameliorates Exercise-Induced Fatigue in Trained Mice. Molecules 19(3): 2793-2807.

### Experiment of Endurance Exercise

The experiment is to evaluate the effect of 4-week supplement of the SA-03 strain of Lactobacillus salivarius of the present invention on the performance of endurance exercise. One week earlier before the experiment (the twenty-first day), and thirty minutes later after the mice were fed with the SA-03 strain of Lactobacillus salivarius of the present invention, let the mice swim in a vat 28 cm in diameter and 25 cm in depth with water at a temperature of 27±1° C. for swimming adaption. On the thirty-first day, undertake exhaustive swimming tests of the mice (5% body weight of the mouse, Wu et al., 2013). The mice fast for 12 hours before swimming. In the experiment, each of the mice swims in a water vat singly. Then, place the mouse in the water vat, and force the mouse to swim. The water in the vat is controlled to be at a temperature of 27±1° C. During the whole experimental process, the four limbs of the tested animal should be kept moving. If the tested animal floats on water surface with the four limbs immobile, a stir bar may be used to stir the water neighboring the tested animal. The experiment of endurance exercise continues until the head of the tested animal has completely sunk into water for 8 seconds.

Analysis of Biochemical Indexes after Exercise Challenge

In order to evaluate the effect of the SA-03 strain of Lactobacillus salivarius of the present invention on post-exercise biochemical indexes and fatigue-related biochemical indexes, inspect the variation of the concentration of blood lactate before and after exercise, and calculate the ratio of post-exercise blood lactate and pre-exercise blood lactate. In order to detect the variation of the fatigue-related blood lactate, feed the mice with the SA-03 strain of Lactobacillus salivarius of the present invention on the day of the swimming test (the thirty-third day), and draw blood 30 minutes later. Next, let the mice swim in water at a temperature of 27±1° C. without carrying a burden for 10 minutes. Next, draw 0.2 mL of blood immediately after swimming and after a 20-minute break. Blood drawing is totally undertaken at three timings for blood lactate analysis.

Further is also inspected the variation of the concentration of the blood urea nitrogen (BUN) of all the groups of mice. On the day of the swimming test (the thirty-fifth day), feed the mice with the SA-03 strain of Lactobacillus salivarius of the present invention. Thirty minutes later, let the mice swim in water for 90 minutes without carrying a burden. After a 60-minute break, draw 0.3 mL blood from the mice for BUN concentration analysis. Further, use a portion of samples to analyze the activity of creatine kinase (CK), which may functions as an index of muscle injury, whereby to learn whether the SA-03 strain of Lactobacillus salivarius of the present invention can prevent from exercise-induced muscle injury. The serum samples are analyzed in an automated hematology analyzer (Hitachi 7060, Hitachi, Tokyo, Japan). The related indexes are reported in the following papers: (Huang C C, Hsu M C, Huang W C, Yang H R, Hou C C. (2012). Triterpenoid-rich extract from Antrodia camphorata improves physical fatigue and exercise performance in mice. Evid Based Complement Alternat Med. 2012:364741); (Wang S Y, Huang W C, Liu C C, Wang M F, Ho C S, Huang W P, Hou C C, Chuang H L, Huang C C. (2012). Pumpkin (Cucurbita moschata) fruit extract improves physical fatigue and exercise performance in mice. Molecules 17(10):11864-11876); (Wu R E, Huang W C, Liao C C, Chang Y K, Kan N W, Huang C C. (2013). Resveratrol protects against physical fatigue and improves exercise performance in mice. Molecules 18(4):4689-4702); (Su K Y, Yu C Y, Chen Y W, Huang Y T, Chen C T, Wu H F, Chen Y L. (2014). Rutin, a flavonoidand principal component of Saussurea involucrata, attenuates physical fatigue in a forced swimming mouse model. Int J Med Sci. 11(5):528-537); (Yeh T S, Chuang H L, Huang W C, Chen Y M, Huang C C, Hsu M C. (2014). Astragalus membranaceus Improves Exercise Performance and Ameliorates Exercise-Induced Fatigue in Trained Mice. Molecules 19(3):2793-2807.

### Analysis of Glycogen Content in Liver and Muscle

The experiment is to evaluate the influence of the SA-03 strain of Lactobacillus salivarius of the present invention on the content of glycogen, which is an important material for energy storage in animal bodies. After a 90-minute swimming, let all the animals take a 2-day break. On the thirty-seventh day of the experiment, sacrifice all the animals 30 minutes later after the last feed, and draw blood for analysis. Further, sample the livers and the muscles in the calves of the hindlimbs. Next, the samples are cleaned with saline, dried and weighed. Next, cut off a portion of each sample, and freeze it at a temperature of −80° C. for the succeeding analysis of glycogen content. The analysis directly quantitatively measures glycogen according to the chemical analysis method used by Chamberland and Rioux (Chamberland V, Rioux P. (2010). Not only students can express alcohol dehydrogenase: goldfish can too! Adv Physiol Educ 34(4):222-227). Add the tested tissue to a tissue homogenate having 5 times the volume of the tested tissue. Next, use a bullet blender (Next Advance, Cambridge, Mass., USA) to homogenize the tissue. Next, add the tissue homogenate to a micro centrifugal tube, and centrifugalize the tissue homogenate at a temperature of 4° C. and a centrifugal force of 12,000×g for 15 minutes. Next, take the supernatant extract, and directly undertake the glycogen content analysis mentioned by Huang (Huang W C, Lin C I, Chiu C C, Lin Y T, Huang W K, Huang H Y, Huang C C. (2014). Chicken essence improves exercise performance and ameliorates physical fatigue. Nutrients. 6(7):2681-2696). Further, use the standard glycogen samples available in the market (Glycogen Sigma) to make a calibration curve, whereby to calculate the glycogen storage in livers and muscles of the mice in different groups and learn the variation thereof.

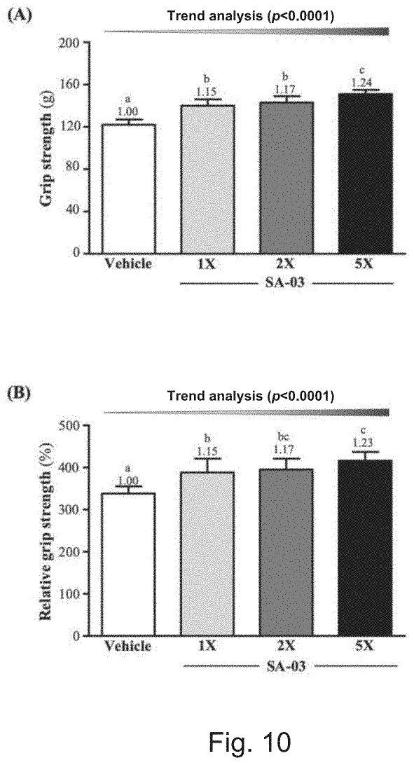

Refer to FIG. 10 for the experiment result of the effect of the SA-03 strain of Lactobacillus salivarius of the present invention on the forelimb grip strength of the mice. FIG. 10A shows the absolute forelimb grip strength. The absolute grip strength is likely to be influenced by the weight of an individual mouse. Therefore, the experiment further calculates the relative grip strengths of all the groups, wherein relative grip strength=forelimb grip strength/body weight×100%. The relative grip strengths are shown in FIG. 10B, wherein if the letters (a, b, c) above the bar chart are different, it indicates significant difference (p<0.05). It is learned from FIG. 10: in comparison with the blank control group, the forelimb grip strengths of the mice are respectively increased by 15%, 17% and 23% in the 1× group, the 2× group and the 5× group, which are respectively fed with 1 time, twice, 5 times the recommended dosage of the SA-03 strain of Lactobacillus salivarius of the present invention. Therefore, the SA-03 strain of Lactobacillus salivarius of the present invention can effectively enhance the forelimb grip strength of the mice. The statistical trend analysis further shows that the enhancement effect of the forelimb grip strength by the present invention has a significant dosage effect (Trend analysis, p<0.0001). In other words, the more the supplement of the SA-03 strain of Lactobacillus salivarius of the present invention, the more the increase of the forelimb strength.

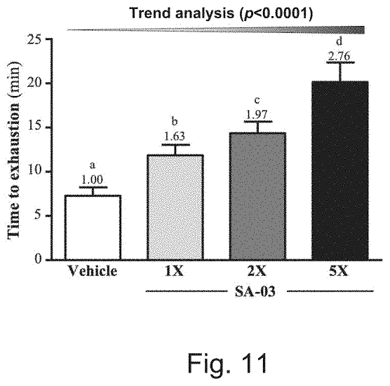

Refer to FIG. 11 for the experiment result of the effect of the SA-03 strain of Lactobacillus salivarius of the present invention on endurance of the mice in the exhaustive swimming tests. If the letters (a, b, c, d) above the bar chart are different, it indicates significant difference (p<0.05). In comparison with the blank control group, the swimming times of the exhaustive swimming tests of the 1× group, the 2× group and the 5× group, which are respectively fed with 1 time, twice, 5 times the recommended dosage of the SA-03 strain of Lactobacillus salivarius of the present invention, are respectively increased by 63%, 97%, and 176%. The statistical trend analysis further shows that the enhancement effect of endurance by the present invention has a significant dosage effect (Trend analysis, p<0.0001). In other words, the more the supplement of the SA-03 strain of Lactobacillus salivarius of the present invention, the longer the swimming time of the exhaustive swimming tests.

Refer to Table. 3 for the effect of the SA-03 strain of Lactobacillus salivarius of the present invention on releasing muscle fatigue of the mice, wherein all the values are expressed in form of Mean±SD, and wherein different letters (a, b, c, d) in the same row indicate significant difference (p<0.05). It is learned from Table. 3: in comparison with the blank control group, the concentrations of the blood lactate of the mice in the 1× group, the 2× group and the 5× group, which are respectively fed with 1 time, twice, 5 times the recommended dosage of the SA-03 strain of Lactobacillus salivarius of the present invention, are significantly decreased. The statistical trend analysis further shows that the effect of reducing post-swimming blood lactate by the present invention has a significant dosage effect (Trend analysis, p<0.0001). In other words, the more the supplement of the SA-03 strain of Lactobacillus salivarius of the present invention, the lower the concentration of post-exercise blood lactate of the mice.

TABLE 3
Effect of Supplementing the SA-03 Strain of the Present Invention on
Variation of Blood Lactate Concentration

| | | | | | Statistical |
|---|---|---|---|---|---|
| Timing of | Blank control | | | | trend |
| drawing blood | group | 1X | 2X | 5X | analysis |
| | Lactate concentration (mmol/L) | | | | |
| Pre-swimming | 3.41 ± 0.69 | 3.34 ± 0.56 | 3.30 ± 0.85 | 3.50 ± 0.37 | 0.6087 |
| (A) | | | | | |
| Post-swimming | 8.73 ± 0.65 ^{d} | 7.53 ± 0.90 ^{c} | 6.65 ± 0.85 ^{b} | 5.78 ± 0.64 ^{a} | <0.0001 |
| (B) | | | | | |
| 20 minutes later | 6.84 ± 0.48 ^{c} | 5.85 ± 1.08 ^{b} | 5.40 ± 0.71 ^{b} | 4.53 ± 0.89 ^{a} | <0.0001 |
| after swimming | | | | | |
| (C) | | | | | |
| | Ratio of generated lactate and Ratio of eliminated lactate | | | | |
| Ratio of | 2.64 ± 0.47 ^{c} | 2.30 ± 0.41 ^{bc} | 2.13 ± 0.57 ^{b} | 1.68 ± 0.30 ^{a} | <0.0001 |
| generated | | | | | |
| lactate = B/A | | | | | |
| Ratio of | 0.21 ± 0.07 | 0.22 ± 0.10 | 0.19 ± 0.08 | 0.22 ± 0.11 | 0.9901 |
| eliminated | | | | | |
| lactate = (B − C)/B | | | | | |

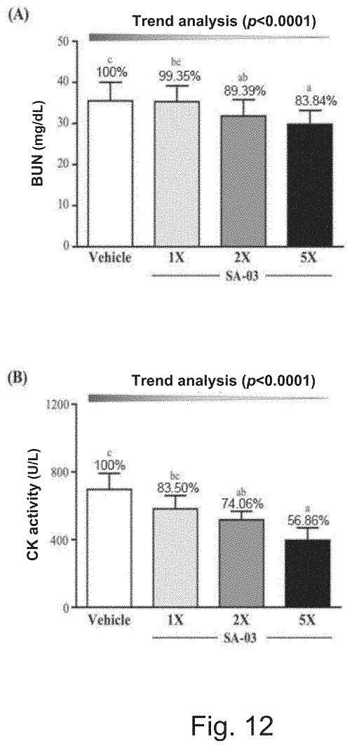

Refer to FIG. 12A for the variation of BUN (blood urea nitrogen) concentrations of all the groups of mice after a single cycle of 90-minute swimming without carrying burden and a 60-minute break, wherein if the letters (a, b, c) above the bar chart are different, it indicates significant difference (p<0.05). The BUN concentrations of the blank control group, the 1× group, the 2× group and the 5× group, which are respectively fed with 1 time, twice, 5 times the recommended dosage of the SA-03 strain of Lactobacillus salivarius of the present invention, are respectively 35.5±4.5, 35.3±3.9, 31.8±4.0, and 29.8±3.4. In comparison with the blank control group, the BUN concentrations of the 2× group and the 5× group are respectively significantly decreased by 10.61% (p=0.0416) and 16.16% (p=0.0027). The statistical trend analysis further shows that the effect of reducing BUN concentration by the present invention has a significant dosage effect (Trend analysis, p<0.0001). In other words, the more the supplement of the SA-03 strain of Lactobacillus salivarius of the present invention, the lower the BUN concentration of the post-swimming mice.

Refer to FIG. 12B for the variation of CK (creatine kinase) activities of all the groups of mice after a single cycle of 90-minute swimming without carrying burden and a 60-minute break, wherein if the letters (a, b, c) above the bar chart are different, it indicates significant difference (p<0.05). In comparison with the blank control group, the CK activities of the 1× group, the 2× group and the 5× group are respectively significantly decreased by 16.50% (p=0.0019), 25.94% (p<0.0001) and 43.14% (p<0.0001). The statistical trend analysis further shows that the effect of reducing CK activities by the present invention has a significant dosage effect (Trend analysis, p<0.0001). In other words, the more the supplement of the SA-03 strain of Lactobacillus salivarius of the present invention, the lower the CK activity of the post-swimming mice. From the abovementioned experiments, it is learned: the supplement of the SA-03 strain of Lactobacillus salivarius of the present invention can reduce muscle fatigue and enhance muscle strength and physical performance.

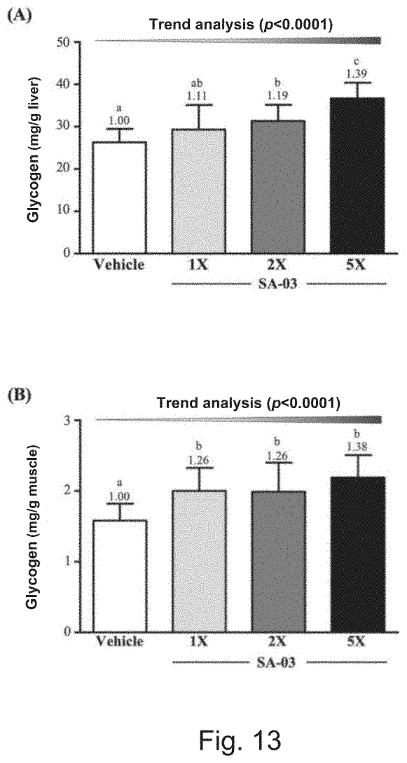

Refer to FIG. 13 for the effect of the SA-03 strain of Lactobacillus salivarius of the present invention on increasing glycogen content, wherein if the letters (a, b, c) above the bar chart are different, it indicates t significant difference (p<0.05). In the experiment, 30 minutes later after the last feed, the four groups of mice are sacrificed. Next, sample the livers and muscle tissues of calves for glycogen content analysis. FIG. 13A shows the experiment results of the glycogen contents in livers. The glycogen contents in the livers of the blank control group, the 1× group, the 2× group and the 5× group, which are respectively fed with 1 time, twice, 5 times the recommended dosage of the SA-03 strain of Lactobacillus salivarius of the present invention, are respectively 26.32±3.14, 29.33±5.83, 31.35±3.82, and 36.69±3.69 (mg/g liver). In comparison with the blank control group, the glycogen contents in the livers of the 2× group and the 5× group are respectively significantly decreased by 1.19 times (p=0.0120) and 1.39 times (p<0.0001). FIG. 13B shows the experiment results of the glycogen contents in the muscle tissues of calves. The glycogen contents in the muscle tissues of the blank control group, the 1× group, the 2× group and the 5× group, which are respectively fed with 1 time, twice, 5 times the recommended dosage of the SA-03 strain of Lactobacillus salivarius of the present invention, are respectively 1.58±0.24, 2.00±0.33, 1.99±0.41, 2.19±0.32 (mg/g muscle). In comparison with the blank control group, the glycogen contents in the muscle tissues of the 1× group, the 2× group and the 5× group are respectively significantly increased by 1.26 times (p=0.0084), 1.26 times (p<0.0098) and 1.38 times (p=0.0002). The statistical trend analysis further shows that the effect of increasing glycogen content by the present invention has a significant dosage effect (Trend analysis, p<0.0001). In other words, the more the supplement of the SA-03 strain of Lactobacillus salivarius of the present invention, the higher the glycogen content in the livers and muscle tissues of the mice.

It should be noted: The functionality of lactic acid bacteria to health is not based on the species of bacteria but dependent on the specificities of strains. The strains favorable to health are called the probiotics (Guidelines for the evaluation of probiotics in food; Report of joint FAO/WHO working group on drafting guidelines for the evaluation of probiotics in food; London Ontario, Canada April 30 and May 1, 2002:1-7). According to a paper published in FOOD AND AGRICULTURAL IMMUNOLOGY 2019 by Ren, et al. (2019, VOL. 30, NO. 1, 281-295), it is found: the CICC 23174 strain of L. salivarius can induce the Th1 reaction and regulate immunity. However, the SA-03 strain of Lactobacillus salivarius of the present invention can induce Treg cells to generate the cytokine IL-10. Thus, the SA-03 strain of Lactobacillus salivarius of the present invention can inhibit the immune reaction of Th1 and Th2 and alleviate allergy and asthma. Therefore, the SA-03 strain of Lactobacillus salivarius of the present invention has its specificity.

In conclusion, the SA-03 strain of Lactobacillus salivarius of the present invention has the physiologically active effects: anti-oxidation, oral health, immunity regulation and exercise promotion. Therefore, the SA-03 strain of Lactobacillus salivarius of the present invention may be in form of a physiologically-acceptable composition or a pharmaceutically-acceptable composition to realize the abovementioned effects.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. A composition for at least one of anti-oxidation, oral health, immunity regulation and exercise promotion, comprising:
an isolated lactic acid bacteria strain, wherein the lactic acid bacteria strain includes an SA-03 strain of Lactobacillus salivarius, and the SA-03 strain of Lactobacillus salivarius is deposited in a Deposition No. of CGMCC No. 19519 in China General Microbiological Culture Collection Center (CGMCC); and
an excipient, diluent or carrier.

2. The composition for at least one of anti-oxidation, oral health, immunity regulation and exercise promotion according to claim 1, wherein the lactic acid bacteria strain is an active strain.

3. The composition for at least one of anti-oxidation, oral health, immunity regulation and exercise promotion according to claim 1, wherein the excipient, diluent or carrier is a food.

4. The composition for at least one of anti-oxidation, oral health, immunity regulation and exercise promotion according to claim 3, wherein the food is fermented milk, yoghurt, cheese, a milk drink, powdered milk, tea, coffee, a tooth-cleaning candy, a functional drink, or a combination thereof.

5. The composition for at least one of anti-oxidation, oral health, immunity regulation and exercise promotion according to claim 1, wherein the excipient, diluent or carrier is a pharmaceutically-acceptable excipient, diluent or carrier.

6. The composition for at least one of anti-oxidation, oral health, immunity regulation and exercise promotion according to claim 5, which is in form of an oral dosage.

7. The composition for at least one of anti-oxidation, oral health, immunity regulation and exercise promotion according to claim 6, wherein the oral dosage is in form of a tablet, a capsule, a solution, or a powder.

8. The composition for at least one of anti-oxidation, oral health, immunity regulation and exercise promotion according to claim 1, wherein the excipient or the diluent is tooth paste, tooth powder, mouthwash, a breath freshening spray, a fluoridizing agent, a false-tooth cleaning agent, a pet tooth cleaning gum, or a hairball remedy gel.

9. The composition for at least one of anti-oxidation, oral health, immunity regulation and exercise promotion according to claim 1, wherein the carrier is a tooth brush, an interdental brush, dental floss, an oral swabstick, or a pet dental chew bone.

10. The composition for at least one of anti-oxidation, oral health, immunity regulation and exercise promotion according to claim 1, wherein the lactic acid bacteria strain is cultured in a medium; the medium includes at least one of carbon sources and nitrogen sources; the carbon source includes glucose, fructose, lactose, sucrose, maltose, galactose, mannose, trehalose, starch, molasses, potato starch, corn starch, malt extract, maltodextrin or combinations thereof; the nitrogen source includes (NH₄)₂SO₄, (NH₄)₃PO₄, NH₄NO₃, NH₄Cl, casamino acid, urea, peptone, polypeptone, tryptone, meat extract, yeast extract, yeast powder, milk, soybean flour, whey or combinations thereof.

11. The composition for at least one of anti-oxidation, oral health, immunity regulation and exercise promotion according to claim 1, wherein the lactic acid bacteria strain is cultured in a medium; the medium includes 2-5% of a mixture of glucose and maltodextrin.

12. The composition for at least one of anti-oxidation, oral health, immunity regulation and exercise promotion according to claim 1, wherein the lactic acid bacteria strain is cultured in a medium; the medium includes at least one of 5-30% milk and 1-10% soybean flour.

13. A use of a composition with a strain of a lactic acid bacterium for at least one of anti-oxidation, oral health, immunity regulation and exercise promotion comprising administering to a subject the composition, wherein the composition comprises:
an isolated lactic acid bacteria strain, wherein the lactic acid bacteria strain includes an SA-03 strain of Lactobacillus salivarius, and the SA-03 strain of Lactobacillus salivarius is deposited in a Deposition No. of CGMCC No. 19519 in China General Microbiological Culture Collection Center (CGMCC); and
an excipient, diluent or carrier.

14. The use of the composition with the strain of the lactic acid bacterium for at least one of anti-oxidation, oral health, immunity regulation and exercise promotion according to claim 13, wherein the lactic acid bacteria strain is an active strain.

15. The use of the composition with the strain of the lactic acid bacterium for at least one of anti-oxidation, oral health, immunity regulation and exercise promotion according to claim 13, wherein the excipient, diluent or carrier is a food.

16. The use of the composition with the strain of the lactic acid bacterium for at least one of anti-oxidation, oral health, immunity regulation and exercise promotion according to claim 15, wherein the food is fermented milk, yoghurt, cheese, a milk drink, powdered milk, tea, coffee, a tooth-cleaning candy, a functional drink, or a combination thereof.

17. The use of the composition with the strain of the lactic acid bacterium for at least one of anti-oxidation, oral health, immunity regulation and exercise promotion according to claim 13, wherein the excipient, diluent or carrier is a pharmaceutically-acceptable excipient, diluent or carrier.

18. The use of the composition with the strain of the lactic acid bacterium for at least one of anti-oxidation, oral health, immunity regulation and exercise promotion according to claim 17, which is in form of an oral dosage.

19. The use of the composition with the strain of the lactic acid bacterium for at least one of anti-oxidation, oral health, immunity regulation and exercise promotion according to claim 18, wherein the oral dosage is in form of a tablet, a capsule, a solution, or a powder.

20. The use of the composition with the strain of the lactic acid bacterium for at least one of anti-oxidation, oral health, immunity regulation and exercise promotion according to claim 13, wherein the excipient or the diluent is tooth paste, tooth powder, mouthwash, a breath freshening spray, a fluoridizing agent, a false-tooth cleaning agent, a pet tooth cleaning gum, or a hairball remedy gel.

21. The use of the composition with the strain of the lactic acid bacterium for at least one of anti-oxidation, oral health, immunity regulation and exercise promotion according to claim 13, wherein the carrier is a tooth brush, an interdental brush, dental floss, an oral swab stick, or a pet dental chew bone.

22. The use of the composition with the strain of the lactic acid bacterium for at least one of anti-oxidation, oral health, immunity regulation and exercise promotion according to claim 13, wherein the lactic acid bacteria strain is cultured in a medium; the medium includes at least one of carbon sources and nitrogen sources; the carbon source includes glucose, fructose, lactose, sucrose, maltose, galactose, mannose, trehalose, starch, molasses, potato starch, corn starch, malt extract, maltodextrin or combinations thereof; the nitrogen source includes (NH₄)₂SO₄, (NH₄)₃PO₄, NH₄NO₃, NH₄Cl, casamino acid, urea, peptone, polypeptone, tryptone, meat extract, yeast extract, yeast powder, milk, soybean flour, whey or combinations thereof.

23. The use of the composition with the strain of the lactic acid bacterium for at least one of anti-oxidation, oral health, immunity regulation and exercise promotion according to claim 13, wherein the lactic acid bacteria strain is cultured in a medium; the medium includes 2-5% of a mixture of glucose and maltodextrin.

24. The use of the composition with the strain of the lactic acid bacterium for at least one of anti-oxidation, oral health, immunity regulation and exercise promotion according to claim 13, wherein the lactic acid bacteria strain is cultured in a medium; the medium includes at least one of 5-30% milk and 1-10% soybean flour.
